(19) United States
(12) Patent Application Publication
(10) Pub. No.: US 2005/0136124 A1
(43) Pub. Date: Jun. 23, 2005

(54) High-yield extraction of osteoinductive agents from DBM and methods for measuring the same

(76) Inventors: Barbara E. Blum, Memphis, TN (US); Charles W. Prince, Birmingham, AL (US)

Correspondence Address: WRIGHT MEDICAL TECHNOLOGY, INC., 5677 AIRLINE ROAD, ARLINGTON, TN, 38002-9501, US

(21) Appl. No.: 10/743,620

(22) Filed: Dec. 22, 2003

(51) Int. Cl. A61K 35/32

(52) U.S. Cl. 424/549

(57) ABSTRACT

This invention is related to methods of extracting and measuring the presence of BMP in DBM. In particular, by omitting the final dialysis step, the measured BMP yield can be dramatically increased.

### FIELD OF THE INVENTION

This invention is related to methods of extracting and measuring the presence of BMP in DBM.

### BACKGROUND OF THE INVENTION

In 1967, Urist, et al, published the seminal article on the theory of bone induction. This article included a method for decalcifying bone, i.e., making demineralized bone matrix (DBM). DBM is an osteoinductive material (induces bone growth when implanted in an ectopic site of a rodent); predominantly due to bone morphogenetic proteins (BMP) contained within the DBM. Honsawek et al. (2000). It is now known that there are numerous BMPs, e.g., BMP 1-15, which are part of the transforming growth factor-beta (TGF-β) superfamily (Kawabata et al., 2000). BMP-2 has become the most important and widely studied of the BMP family of proteins. There are also other proteins present in demineralized bone matrix (DBM) that are not osteoinductive alone but contribute to bone growth, including fibroblast growth factor-2 (FGF-2), insulin-like growth factor-I and -II (IGF-I and IGF-II), platelet derived growth factor (PDGF), and transforming growth factor-beta 1 (TGF-β1) (Hauschka, et al. 1986; Canalis, et al, 1988; Mohan et al. 1996).

In 1981, Urist received U.S. Pat. No. 4,294,753, the first of his many patents on a process for extracting BMP from DBM. At the time of the Urist '753 patent, BMP was refered to generally. However, as mentioned above, now it is known that there are multiple forms of BMP.

The Urist process became widely adopted and though different users may use different chemical agents from those disclosed in the basic Urist process, the basic layout of the steps of the process remains widely adopted today as one of the main methods of extracting BMP from DBM. See e.g. U.S. Pub 2003/0065392 (2003); U.S. Pub 2002/0197297 (2002). Jefferies, another lead researcher in this field, also uses the Urist process. See U.S. Pat. No. 6,311,690 (2001). In any event, Urist reported that the basic Urist process actually results in generally low yields of BMP per unit weight of DBM. Urist et al (1982).

Therefore, other processes have been attempted, with little improvement.

However, if it were possible to increase the BMP yield of the conventional Urist process, due to widespread experience with the process, that would be highly beneficial. Accordingly, there is room for improvement within the art.

### OBJECTS OF THE INVENTION

It is an object of the invention to increase the BMP-2 yield of conventional BMP extraction methods.

It is an object of the invention to improve our ability to detect BMP-2 in a BMP extract.

These and other objects of the invention are achieved by a process for extracting BMP from DBM, comprising the steps of: providing DBM; mixing the DBM with a cold aqueous solution of a neutral salt and a solubolizing agent for BMP; extracting BMP from the resulting precipitate and liquid fraction without dialyzing either of the resulting precipitate and liquid fraction.

These and other objects of the invention are achieved by a process for extracting BMP from DBM, including the step of retaining molecules having a molecular weight of at least 10 kDa.

### BRIEF DESCRIPTION OF THE FIGURES

FIG. 1 depicts the amount of BMP-2 in DBM extracts as related to Example One.

FIG. 2 depicts the percentage of new bone associated with DBM implants in athymic nude rats vs. amount of BMP-2 in DBM extracts as related to Example Two.

FIG. 3 depicts the percentage of new bone associated with implants of a calcium sulfate/DBM bone graft substitute in athymic nude rats vs. amount of BMP-2 in DBM extracts as related to Example Three.

FIG. 4 depicts the concentration of BMP-2 in DBM extracts, presented as the mean of 16 replicates/lot. Bars represent standard deviation, and CV (%) was calculated as standard deviation÷mean as related to Example Four.

### DETAILED DESCRIPTION OF THE INVENTION

As mentioned, the invention is concerned with increasing the osteoinductive and/or BMP yield of conventional BMP extraction processes. While the osteoinductive potential of a material is predominantly based upon its BMP content and applicants believe their increased osteoinductivity is due to increased BMP levels, applicant's cannot be sure that is the mechanism of their invention. Furthermore, as will be described below, it is not clear whether applicant's process merely makes the detection of already present BMP-2 easier.

The basic Urist process described in U.S. Pat. No. 4,294,753 includes a step just prior to the beginning of optional processing in which the BMP precipitate is dialyzed to remove urea, CaCl2, and other impurities. '753 patent (col. 5, lines 51-57). Urist continued to include this mandatory dialysis step in his series of patents on BMP extraction and the others previously mentioned who extract BMP from DBM using Urist's basic method incorporated this dialysis step. E.g., U.S. Pat. No. 6,311,690 (col. 8, lines 49-51; col. 14, lines 44-47).

This dialysis step was thought to remove impurities that are not conducive to bone growth. However, Applicants have determined that this dialysis step, considered mandatory by Urist, can be omitted, while increasing the BMP yield from a given DBM sample. Note the following Example, which proves this out.

### EXAMPLE ONE

### BMP-2 Level With and Without Dialysis

Proteins were extracted from two 1 g samples of DBM from six lots using a Tris-collagenase solution (1,000 U/mL collagenase). A third sample of DBM from each lot was ‘extracted’ with a Tris buffer solution without collagenase, intended to yield only water-soluble proteins not entrapped within the matrix. All samples were extracted in a 37° C. shaking water bath for 18 hours. Following extraction, one collagenase extract sample from each lot was dialyzed overnight (i.e., conventional Urist method) at 4° C. against glutamic acid-hydrochloride (5 mM) (Mᵣ cut-off≈10 kD).

Extracts were assayed for the osteoinductive protein BMP-2. The levels of BMP-2 in the extracts were detected by a conventional BMP-2 ELISA kit (R&D Systems, Minneapolis, Minn.). The results are detailed in FIG. 1 and indicate that BMP-2 levels were highest in the undialyzed extracts and lowest in the dialyzed collagenase-extracts.

In summary:

Mean BMP-2 Content Using Dialysis: 246 pg/g DBM.

Mean BMP-2 Content Omitting Dialysis: 1459 pg/g DBM.

This mean BMP-2 content is extremely higher than BMP-2 extracted via traditional methods and has not been achieved before.

### EXAMPLE TWO

### New Bone Growth With DBM in Athymic Nude Rat Muscle Pouch Model vs. BMP-2 Levels

Samples (50 mg) of 60 lots of unsterilized DBM were implanted in intermuscular sites in the latissimus dorsi in athymic nude rats (4 implants/rat; n=6/lot). Implants were harvested at 28 days, decalcified, processed, embedded in paraffin, cut from the middle of the specimen, and stained with hematoxylin and eosin (H&E). New bone, as a percentage of total tissue area examined, was measured in ten regions within each specimen, using image analysis software (Bioquant®, Nashville, Tenn.).

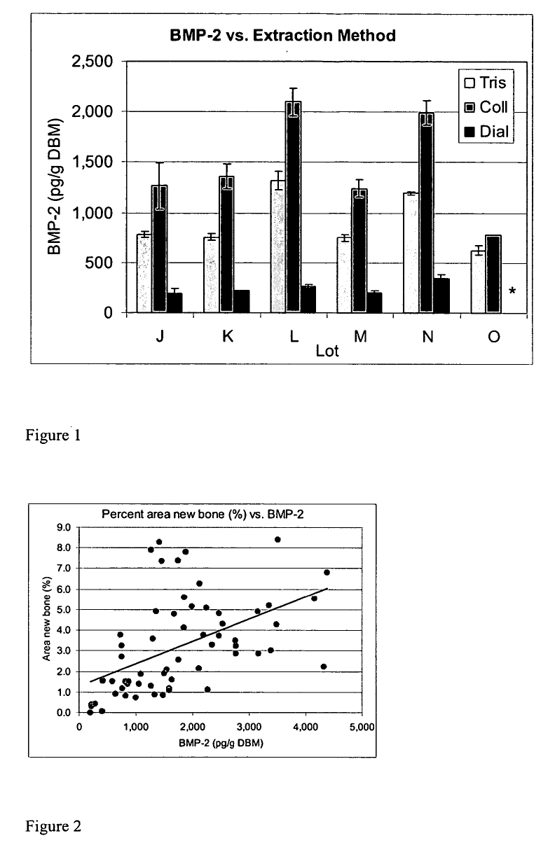

Samples (1 g) from the same lots were also collagenase-extracted and assayed for BMP-2 using commercially available ELISA kits (R&D Systems, Minneapolis, Minn.). Linear regression analysis revealed a strong correlation between BMP-2 and percentage new bone growth (p<0.001; Power=0.996), demonstrating that BMP-2 measured in non-purified (undialyzed) bone extracts is an indicator of osteoinductivity as traditionally defined (i.e., in an ectopic rodent model). See FIG. 2.

### EXAMPLE THREE

### New Bone Growth With a DBM-Containing Bone Graft Substitute in an Athymic Nude Rat Muscle Pouch Model vs. BMP-2 Levels

In study similar to that described in Example 2, samples of a calcium sulfate/DBM bone graft substitute were prepared from 50 lots of DBM and implanted in athymic nude rats. As before, samples were harvested at 28 days, processed for histology, and evaluated for new bone growth. Results were compared to levels of BMP-2, measured as before, in the same 50 lots.

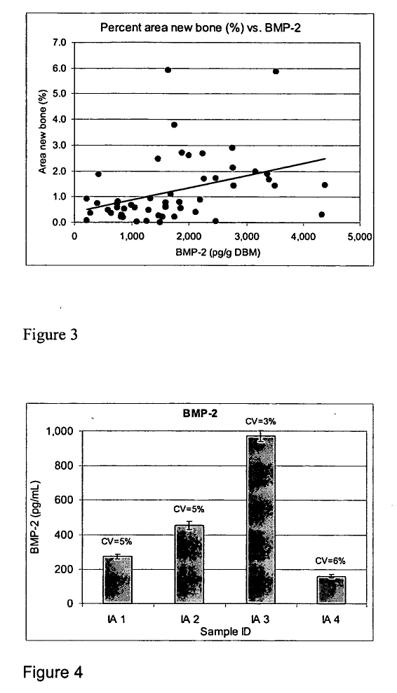

Again, linear regression analysis revealed a strong correlation between levels of BMP-2 and new bone growth seen with the DBM-containing bone graft substitute (p=0.006; Power=0.785). See FIG. 3.

### EXAMPLE FOUR

### Testing to Demonstrate Repeatability

A series of evaluations were conducted to confirm repeatability of results of protein extraction and subsequent assay for BMP-2.

To evaluate intra-assay variability, extract samples from four different lots of DBM were tested within one assay, n=16 wells (replicates)/lot. The mean coefficient of variance of BMP-2 levels (CV, %=standard deviation÷mean) was 5.0±1.2% (range 3-6%). These values were within the range previously observed in a study of 20 replicates/lot conducted by the kit supplier as described in the BMP-2 Quantikine Kit Instructions for Use (range 5.2-7.5%, mean 6.1%). The results of this test are shown in FIG. 4.

In a second study, inter-extraction and inter-assay variability was assessed in one study by extracting samples from five lots of DBM three different times (extracts A-C), and then assaying samples from all three extracts (A-C) seven times (Assays 1-7), resulting in 7×3=21 data points (ie, BMP-2 level) from each lot. Results were evaluated using intra-class correlation. Intra-class correlation (‘Three-between subjects design without replication’), designed to determine both consistency and level of error, revealed that results are highly repeatable. Intra-class correlation (ICC) was calculated as 0.9624, suggesting that the results are very repeatable between different extractions and different assays. Standard error of measurement (SEM) was 46.44 pg/g DBM (on an overall mean of 1645 pg/g DBM), indicating that we can be 95% confident that the values observed with any particular extraction/assay would be within +93 pg/g DBM.

Theory of the Invention

Without wishing to be bound to any particular theory, it is believed that two factors contribute to the increased BMP-2 levels detected when omitting the dialysis step of the basic Urist process. Both factors, however, are based on the fact that it is thought that it is possible that dialysis removes certain small molecules and fragments of molecules from the BMP solution that may have meritorious effects.

For example, first, those molecules may comprise small broken-up BMP-2 molecules. By washing these molecules away via dialysis, ELISA will detect less BMP-2.

Second, it is possible that these small molecules may include yet unidentified or understood low molecular weight molecules that contribute to the ELISA kit detecting BMP-2 in general. In other words, these low molecular weight molecules, may effect cross-reactivity between BMP-2 and the BMP-2 assay kit (ELISA). Thus, by reducing the amount of these molecules present, the ELISA kit will skew the BMP-2 results downward.

The basis for these theories is supported in the literature. In 1979, Urist et al. separated proteins from rabbit DBM in a six-step process which culminated in elution in α-mannoside or ethylene glycol to liberate BMP bound by hydrophobic interaction or carbohydrate recognition (Urist, et al, 1979). DBM was converted to gelatin, extracted in a Tris/collagenase (0.00054%) buffer, and dialyzed in 0.1% ethylene glycol or PBS/ethylene glycol (Mᵣ cut-off≈2 kDa). Co-precipitates were also extracted and dialyzed again. Following elution of samples in α-mannoside or ethylene glycol to liberate BMP bound by hydrophobic interaction or carbohydrate recognition, sodium dodecyl sulfate (SDS)-gel electroporesis produced bands at 14.3, 21, 43, 68 and 94 kDa (α-mannoside only), with the 68 kDa band being the densest. Elution with α-mannoside resulted in BMPs and other glycoproteins with five molecular weights between 14.3 and 94 kDa. Four glycoproteins/BMPs between 14.3 and 63 kDa were eluted with ethylene glycol. Two glycoproteins/BMPs between 14.3 and 21 kDa were found with co-precipitation with calcium phosphate. When implanted within double-walled cellulose acetate chambers in the anterior abdominal wall of allogeneic rabbits, all three mixtures had comparable BMP activity. Amino acid analysis identified aspartic acid and glutamic acid as the predominant residues. There was also an abundance of leucine, valine, proline, and lysine, and some half-cysteine; no hydroxyproline was evident. However, what was important was the conclusion that there was the possibility that the biologic activity of the BMP may be a function of a protein aggregate. Urist et al, at 1831. The nature of that aggregate was left to further research. Id.

Mizutani et al. extracted proteins from 10 kg bovine DBM with a 4M Guanidine-hydrochloride solution and purified the solution with several dialysis (Mᵣ cut-off≈10 kDa), precipitation, and chromatography steps (diethylaminoethyl, DEAE; and carboxymethylcellulose, CMC) (Mizutani and Urist, 1982). The resultant purified solution contained protein peaks at 14, 17.5, 24, and 34 kD, as well as 57 and 72 kD. Implantation of reconstituted lyophilized proteins from each of these bands in the thighs of CBA-strain mice demonstrated osteoinductivity of the 17.5 kDa protein. The 14 and 24 kDa proteins did not have biologic activity alone, but the 24 kD protein showed high activity when implanted with the 17.5 kDa protein and other components. The 57 and 72 kD proteins also showed some activity when implanted with the proteins between 17.5 and 34 kDa present.

Accordingly, while some smaller molecular weight molecules did not have osteoinductive potential when alone, they did when combined with others. Mizutani et al. attributed this to the fact that certain molecular weight proteins may effect the solubility of other molecular weight proteins. Mizutani et al., at 222. We call this cross-reactivity.

Urist et al. then extracted proteins from DBM into 6M Urea solution or 4M guanidine-hydrochloride solution and further purified the fluid by precipitation and dialysis in several different urea solutions (Urist et al, 1982). A lyophilized precipitate was dissolved in 6M urea and filtered through either CM-cellulose or DEAE-cellulose (negatively and positively charged, respectively), to bind non-BMP proteins. The final material was obtained by dialyzing the supernatant and lyophilizing the precipitate. After filtered or unfiltered extracts were separated by CL-6B Sepharose gel into nine fractions, only the sixth fraction (10-30 kDa; peak 22 kDa) induced bone growth in the thigh of mice. Urist et al. concluded that BMP is probably a low molecular weight molecule, Urist et al., at 226, whose main components have a molecular weight of <25 kDa. Id. at 230-31.

Urist et al. then demineralized 10 kg of bovine bone and purified the Gu-HCl protein extract by repeated dialysis and precipitation (Urist et al, 1984). After separating Urea-soluble proteins into four bands (17, 17.5, 18.5, 22 kDa) by hydroxyapatite chromatography, they evaluated the osteoinductivity of each in a muscle-pouch model in Swiss mice, and a skull trephine model in monkeys, dogs, and rats. The 18.5±0.5 kDa fraction, which showed properties of an acidic polypeptide and may have included hydroxyproline and possibly V-carboxyglutamic acid, was the only fraction associated with new bone growth in the animal assays when implanted without other protein fractions.

Sampath and Reddi compared the amount of proteins yielded from rat DBM at 16 hours following extraction with 8M urea, 4M Gu-HCl, and 1% sodium dodecyl sulfate (SDS) (Sampath and Reddi, 1981). All methods resulted in insoluble matrices with no osteoinductive properties, although 8M Urea was effective in eliminating osteoinductivity only when combined with 1 M NaCl. Each residue and supernatant was dialyzed against water (molecular weight cut-off≈3.5 kDa) and lyophilized, either separately or together. Separated components were recombined before implantation. When implanted subcutaneously in Long-Evans rats for 12 days, extracts and residues recombined before lyophilization were more osteoinductive than samples recombined mechanically after lyophilization, as determined by calcium content and alkaline phosphatase activity of the explants. Gel filtration of guanidine-hydrochloride extracts indicated that the osteoinductive proteins were smaller than 50 kDa.

Thus we have concluded that based upon our review of this previous research, small molecular weight molecules (e.g. less than about 10 KDa) may exist in pre-dialyzed solution that could contribute to osteoinductivity of the BMP product if left in solution, but have been previously washed away by dialysis. Furthermore, as the above research has shown that some of these low molecular weight molecules only work in combination with certain other proteins, it is highly possible that these small molecular weight molecules may contribute to the osteoinductivity of BMP-2, its solubility, and/or its cross-reactivity with the BMP-2 assay. Therefore, it becomes preferable to retain these small molecular weight molecules rather than remove them inadvertently in the dialysis step.

While the invention has been described with respect to certain preferred methods and theories, the scope of the invention is bound by the claims and equivalents thereof.

### REFERENCES

Canalis et al., Isolation of Growth Factors from Adult Bovine Bone Matrix, Calcif. Tissue Int. 43(6): 346-351 (1988).
Hauschka et al., Growth Factors in Bone Matrix. Isolation of Multiple Types of Affinity Chromtography on Heparin Sepharose. J. Blol. Chem. 261(27): 12665-12674 (1986).
Honsawek et al., BMP-4 Content in Human Deminerilzed Bone Matrix Correlates With Osteoinductivity, Trans. Orthop. Res. Soc., Vol. 28, Paper #0020, (2003).
Kawabata et al., Bone Morphogenetic Proteins, In: Skeletal Growth Factors (E. Canalis, ed.) Lippincott, Williams and Wilkens, Chap. 19, (2000).
Mizutani et al., The Nature of Bone Morphogenetic Protein (BMP) Fractions Derived From Bovine Bone Matrix Gelatin, Clin. Orthop. Rel. Res., 171:213-223 (1982).
Mohan et al., Therapeutic Potential of TGF-β, BMP and FGF in the Treatment of Bone Loss, In: Principles of Bone Biology (Bilezekian J P, Raisz L G, eds.) Academic Press, pp. 1111-1123 (1996).
Sampath et al., Dissociative Extraction and Reconstitution of Extracellular Matrix Components Involved in Local Bone Differentiation, Proc. Natl. Acad. Sci. USA 78:7599:7603 (1981).
Urist et al., The Bone Induction Principal, Clin. Orthop. Rel. Res., 53:243-283 (1967).
Urist et al., Solubilized and Insolubilized Bone Morphogenic Protein, Proc. Natl. Acad. Sci. USA 76(4):1828-1832 (1979).
Urist et al., A Bovine Low Molecular Weight Bone Morphogenetic Protein (BMP) Fraction, Clin. Orthop. Rel. Res., 162:219-232 (1982).
Urist et al., Bone Cell Differentiation and Growth Factors, Science, 220:680-686 (1983).

1. A process for extracting BMP from DBM, comprising the steps of:
providing DBM;
mixing a first portion of said DBM with an aqueous solution of a neutral salt and a solubilizing agent for BMP to thereby obtain a liquid fraction; and
assaying BMP from the resulting liquid fraction with an ELISA kit to determine osteoinductivity without dialyzing said liquid fraction.

2. The process of claim 1, further comprising the steps of:
taking a second portion of said DBM and determining its osteoinductivity by implantation in an athymic nude rat;
the osteoinductivities of said first and second portions being substantially the same.

3. The process of claim 1, wherein the solubilizing agent is collagenase.

4. A process for extracting BMP from DBM, including the step of retaining molecules having a molecular weight of less than 10 kDa.

5. A process for determining osteoinductivity of DBM via in vitro assay of BMP comprising:
providing DBM;
solubilizing BMP from the DBM to thereby obtain an unfiltered BMP containing solution; and
assaying the unfiltered BMP containing solution to determine a level of a selected BMP in the unfiltered BMP containing solution.

6. The process of claim 5, further comprising comparing the level of the selected BMP to data correlating BMP level with osteoinductivity to thereby predict osteoinductivity of the DBM.

7. The process of claim 5, wherein the assay of the selected BMP is conducted with an ELISA kit.

8. The process of claim 5, wherein molecules having a molecular weight of less than 10 kDa are retained in the BMP containing solution during the assay for BMP.

9. The process of claim 5, wherein the selected BMP is BMP-2.

10. The process of claim 5, wherein the level of the BMP-2 in the BMP containing solution is greater than about 1400 pg/g DBM.

11. The process of claim 5, further comprising:
implanting a portion of the DBM into at least one rat to determine a level of new bone growth induced by the DBM in the at least one rat, and correlating the level of new both growth in the at least one rat with the level of the selected BMP in the unfiltered DBM containing solution to thereby obtain data correlating BMP level of the DBM with osteoinductivity of the DBM.
